(19) United States
(12) Patent Application Publication
(10) Pub. No.: US 2009/0191631 A1
(43) Pub. Date: Jul. 30, 2009

(54) 3-D petri-dish for the culture and studies of cells

(76) Inventor: Reimhard Bornemann, Bielefeld (DE)

Correspondence Address: Joerg C. Gerlach, 2634 S. 18th Street, Pittsburgh, PA, 15210, US

(21) Appl. No.: 11/810,856

(22) Filed: Jun. 7, 2007

(30) Foreign Application Priority Data
Jul. 10, 2006 (DE) 102006031871.4-41

(51) Int. Cl. C12N 5/06; C12M 1/00

(52) U.S. Cl. 435/395; 435/297.4; 435/288.3

(57) ABSTRACT

A three-dimensional (3-D) culture “Petri-dish” for research in regenerative medicine, biotechnology and clinical translation is described. This 3-D perfusion culture dish is to advance in vitro culture tools from static 2-D to dynamic 3-D perfusion culture. Interwoven hollow fiber capillary membranes divide the “Petri-dish” culture space into a controllable 3-D pattern of different compartments, serving the functions of the organ's larger vasculature. These physically active scaffolds, which can be suitable for cell adhesion or cell aggregate immobilization, offer a supply of cells with high-performance mass exchange including gas supply and under perfusion conditions. In contrast to static and discontinuous medium supply, a dynamic culture can be achieved with continuous or alternating medium supply and integral oxygenation. They provide a more physiologic supply in the cell macro environment, including homeostasis of oxygen, pH, nutrition, soluble factors, and gradients of metabolites for the cells. Also, medium perfusion can be achieved. Consequently the invention was made for cultures at tissue density, especially stem cells and support cells, which strive to create their own stem cell niche.

### FIELD OF THE INVENTION

Nature uses self-assembly, on the nano-micro- and macroscopic level to organize molecules and cells into complex tissues. Purpose of the invention was to provide a tool for in vitro cell culture research to study some of the involved physical and biological signals to recapitulate developmental and regenerative processes in cell-, tissue- and organ specific differentiation and morphogenesis. This is of special interest for in vitro stem cell culture.

### BACKGROUND

Stem cells divide into daughter cells that divide into tissue-typical cells, the first daughter cell divides into tissue-typical cells and the second cell retains the stem cell character and remains at the place of origin.

To better understand the control of stem cell differentiation and in vitro proliferation, several areas of research are of interest. Numerous groups are studying signaling at the cellular level. Most experience was gained studying soluble factor- and receptor interactions, and these studies typically base on the use of Petri-dish in vitro culture vessels. Recently, exciting results were achieved studying cell-biomatrix interactions. Although scaffolds provide oversimplified mimics of natural macro environmental cues lacking the essential natural spatial and temporal complexity, these methods move research into three-dimensional (3D) in vitro cultures.

However, physical factors of the cellular macro environment (including oxygen tension, pH, temperature, substrate gradients, medium flow) and 3D cell-cell interactions at tissue densities are poorly understood. One reason for this lack in knowledge is the problem that these factors have to be investigated under high-density tissue perfusion conditions, where physiological cell-cell contacts can be reestablished by the cells. Here, innovative 3D cell perfusion culture vessels may play an important role, e.g. as a tool for studies to help understand these mechanisms.

A 3D environment is of importance for in vitro stem cell research in regenerative medicine.

Stem cells are characterized by asynchronic divisions, the first daughter cell differentiates into tissue-typical cells and the second retains the stem cell character and continues to reside at the stem cell site. To describe the complex interactions with the supporting environment at that site, regenerative medicine research focuses on the concept of the “stem cell niche” (Poser K., Its the ecology, stupid! Nature 2005; 435: 268-270). In ecology, a niche is defined as a specific location where organisms live, what they do and how they interact with the environment. Taking an organism out of that niche often dramatically alters its well-being and behavior.

The term “stem cell niche” in cell biology was introduced by Spradling and Fuller to describe the microenvironment in which stem cells reside in the body (Xie T, Spradling A C. Science 2000; 290:328-330. Xie T, Spradling A C, Kiger A A, Jones D L, Schulz C, Rogers M B, Fuller M T. Science 2001; 294:2542-2545. Science 2000; 290:328-330). Regenerative Medicine research now faces the problem that the current in vitro culture conditions are not sufficient to mimic a stem cell niche as it occurs in the natural environment.

Taking apart the stem cell niche for in vitro culture, interactions involved may be deleterious if stem cells are to fulfill their therapeutic promise, and eventually deploy to grow specialized cells and tissues to replace or regenerate those lost to injury or disease.

Stem cell responses to the microenvironment were already described four decades ago (Wolf N S, Trentin J J. J. Exp. Med. 1968; 127:205-214) on the example of bone marrow stem cells in the bone. Residing in the biomatrix-coated, open porous foam-like hydroxyapatite scaffold of the bone enables hematopoietic stem cells to remain undifferentiated; however, removing them from this environment for research purposes results in rapid differentiation and the loss of their stem cell character. Such an idyllic niche is described for stem cells of the skin-, hair-, intestine-, some specialized regions of the brain, and is in debate for further tissues such as the liver. In addition to soluble mediators/receptors and diffusible biochemicals, the physical anchoring, e.g. through cell-surface receptors plays an important role. The stem cells are typically embedded in supporting cells that literally hold on to the stem cells, lining them up in place and helping to direct the production of daughter cells to differentiate properly.

In the bone marrow, osteoblast cells lining the inner hydroxyapatite bone surface are closely associated with hematopoietic stem cells, encouraging their asymmetric division and regulating their subsequent differentiation into the precursors of blood cells and the immune system (Zang j et al. Nure 2003; 425:836-841. Calvi L M et al. Nature 2003; 425:841-846). In the hippocampus region of the brain, neural stem cells take their cues from both endothelial cells and astrocytes, supporting the cells to stay in the niche (Shen Q et al. Science 2004; 304: 1338-1340) or to develop into neurons (Song H, Stevens C F, Gage F H. Nature 2002; 417:39-44). It appears, that stem cells may even be able to populate their microenvironment with the support cells that they need to thrive, as indicated for neuronal stem cells, giving rise to endothelial cells (Wurmser A E et al. Nature 2004; 160:350-356) or skin stem cells (Blanpain C, Lowry W E, Goeghegan A, Polak L, Fuchs E. Cell 2004; 118:635-648).

Therapeutic Regenerative Medicine applications, currently in development, require removing the stem cells from their niche and placing them into an in vitro situation, e.g. to greatly expand stem cell numbers for clinical therapy. The actual state of the art cell culture vessel is the Petri-dish. Petri-dishes expose the cells to a static 2D environment, over simplifying the complex in vivo situation.

Using more advanced in vitro culture tools, such as a 3D perfuseable “Petri-dish”, and providing a more organized 3D perfusion and co-culture environment, will help to better understand the functions of the stem cell microenvironment, providing clues, e.g., about how to reactivate quiescent stem cells, or send stem cells to different developmental pathways.

Bioreactors for cell culture, or cell culture apparatuses are well known. This includes the use of distinct membrane systems. An example is given in 1990 by R. Cousins (Cell culture apparatus, European Patent Application 0 419 234 A3). Systems with more than one membrane systems, however do not interweave the membranes to an alternating pattern in the cell compartment. All these devices, however, cannot be regarded as “open” systems exhibiting a lid which can be opened at any time and which allows acces to the entire culture. An example of a closed system is given in 1995 by B. P. Amiot (Cell Culture Apparatus, U.S. Pat. No. 5,416,022). An example of a potential open system is given in 1998 by Schmitz et al. (German Patent DE 42 18917 C2), in which a solid structure complete fills out the cell compartment and does not allow cell sampling or cell imaging. Another example of a bioreactor with solid internal structures in the cell compartment is given in 1999 by Creavis (German Patent Application DE 199 19 241 A1). A Petri-dish or a cell culture dish or a cell culture flask are considered as being “open” to allow access to the cell compartment and cell imaging. They exhibit a lid, which can be opened any time during culture to remove cell samples, or to manipulate cells; they are also made of transparent plastic material, thus enabling cell imaging inside the compartment. An example for cell access is described in 2004 by Orwar et al. Method for Electro-permeabilization . . . , US Patent Application US 2004/0023394 A1). These features are not presented by the state of the art bioreactors. Petri-dishes, however, are made for static conditions and for two-dimensional (2D ) cell cultures on the bottom of the dish. We took this as a challenge to develop an innovative Petri-dish, featuring all advantages of both concepts.

The terms used in the following, generally but not exclusively refer to the following: The term membrane system is used to describe an assembly of several hollow fiber capillary membranes run in parallel but connected via a common inlet and outlet. Several of these membrane systems may be used in an interwoven or parallel array. The term culture space is used for the part in the modified Petri-dish in which cells can be cultivated, surrounded by/in between parts of the membrane systems that provide mass exchange for the cells. The term inner block is used for an assembly of all membrane systems and associated culture spaces, cast together into a common structure, which can be placed into a housing, including—as an insert—into the inner chamber of a conventional Petri-dish. The term stem cells applies to animal and/or human stem cells, including adult and embryonal stem cells.

### SUMMARY OF THE INVENTION

The invention pertains to a modified culture dish/flask, particularly a modified Petri-dish for three-dimensional (3D) perfusion cultures for research in cell biology, biotechnology, and clinical translation/application in the field of regenerative medicine and cell application.

An innovative 3D perfusion culture “Petri-dish” for research in regenerative medicine, biotechnology and clinical translation is described.

Several areas of stem cell research focus on the control of in vitro cell differentiation. Physical factors of the cellular macro environment (including oxygen tension, pH, temperature, substrate gradients, medium flow) and 3D cell-cell interactions at tissue densities are poorly understood. One reason for that lack in knowledge is the problem that these factors have to be investigated under high-density tissue perfusion conditions, where physiological cell-cell contacts can be reestablished by the cells. Purpose of the described innovative 3D perfusion culture dish is to advance in vitro culture tools from static 2D to dynamic culture. Characteristic is a decentralized 3D perfuseable culture space for the 3D immobilization of cells at tissue density and the provision of a compartmentalized 3D perfusion- and co-culture space.

Interwoven hollow fiber membranes divide the 3D Petri-dish culture space into a controllable pattern of different compartments, serving the functions of the organ's larger vasculature. These physically active scaffolds provide a supply for the cells with high mass exchange and under perfusion conditions, enabling cell culture at tissue densities. A more physiologic supply in the cell macro environment can be achieved, including homeostasis of oxygen, pH, electrolyte, nutrition, soluble factors, and avoiding gradients of metabolites.

This 3D perfusion culture vessel allows a spontaneous reassembly of co-cultured parenchymal and non-parenchymal cells (e.g. endothelial cells) in vitro and therefore, studies on assembly, regeneration, reparation and function of tissues. The regulatory effects of cell surface signals, soluble factor signals and macro environmental factors can be better addressed in this 3D perfusion culture dish. Applications include biotechnology procedures and the development of therapeutic cell-based interventions in regenerative medicine.

### BRIEF DESCRIPTION OF DRAWINGS

FIG. 1: A 3-D perfusion culture dish according to the invention, with three hollow fiber membrane systems inside a rectangular culture space, and tube connections for perfusion.

FIG. 1 shows a modified Petri-dish, according to the invention. It illustrates the transition from a 2D Petri-dish for static culture to a 3D perfusion culture dish. Perfuseable hollow fiber membranes are included and separated into areas where the various fibers are potted into potting material (such as cast polyurethane) leading into the culture compartment or leaving the culture compartment. The hollow fiber membranes itself divide the culture compartment into compartments which communicate with the flow heads and flow tubes leading out of the dish. The 3D architecture of the hollow fibers leads to a 3D culture space where the cells can be cultivated between, or on the surface of the membranes. The bundling of specific membranes via flow heads and tubes allows the perfusion of the 3D culture space. This modified culture dish is formed by a frame I that runs along the edge of a base plate 2, thereby containing the inner space 11 of the modified Petri-dish. 1 and 2 represent a traditional Petri-dish. The inner space 11 of the modified Petri-dish contains a block 3 that consists of three areas 4, 5a, 5b. The three hollow fiber membrane systems 6a, 6b, 6c successively pass through the areas 4, 5a, 5b. At one part of block 3, one end of the individual hollow fiber membranes of the hollow fiber membrane systems 6a, 6b, 6c are bundled into collective inlets 7a, 7b, 7c that are connected to supply lines 9a and 9b. On the other end, the individual hollow fiber membranes of the hollow fiber membrane systems 6a, 6b, 6c are bundled into collective outlets 8a, 8b, 8c that are connected to outlet lines 10a and 10b. Here, the hollow fiber membrane systems 6a and 6b are arranged in such a way that, in chamber 4, the inflowing substance occurs in counter-directional flow.

To achieve this, the inlet line 9a branches off, whereby one branch is connected to the hollow fiber membrane system 6a, while the other branch runs along frame 1 of the modified Petri-dish to the other side where it is connected with hollow fiber membrane system 6b via connector 7b. Hollow fiber membrane system 6c is independently serviced with substances via inlet 9b and outlet 10b.

According to the invention, only area 4 that is separated within block 3 is used for cell culture. The areas 5a and 5b are cast with plastics, e.g. polymers, e.g. polyurethane, to fixate, and stabilize the hollow fiber membranes of the hollow fiber membrane systems 6a, 6b, 6c and isolate them from each other.

The individual hollow fibers of the hollow fiber membrane systems 6a, 6b, 6c can be designed as tubes, and/or as capillaries. In this example, gas or oxygen can be transported through the independent capillary system 6c, while medium or another fluid can flow through the two connected systems 6a and 6b.

Also, according to the invention, the culture spaces 4 can be closed with a lid or the entire culture dish can be closed with a lid. This lid can prevent medium shift during high flow/pressure operation.

FIG. 2: A modified culture dish according to the invention, with three hollow fiber membrane systems and four rectangular culture spaces, and tube connections for perfusion.

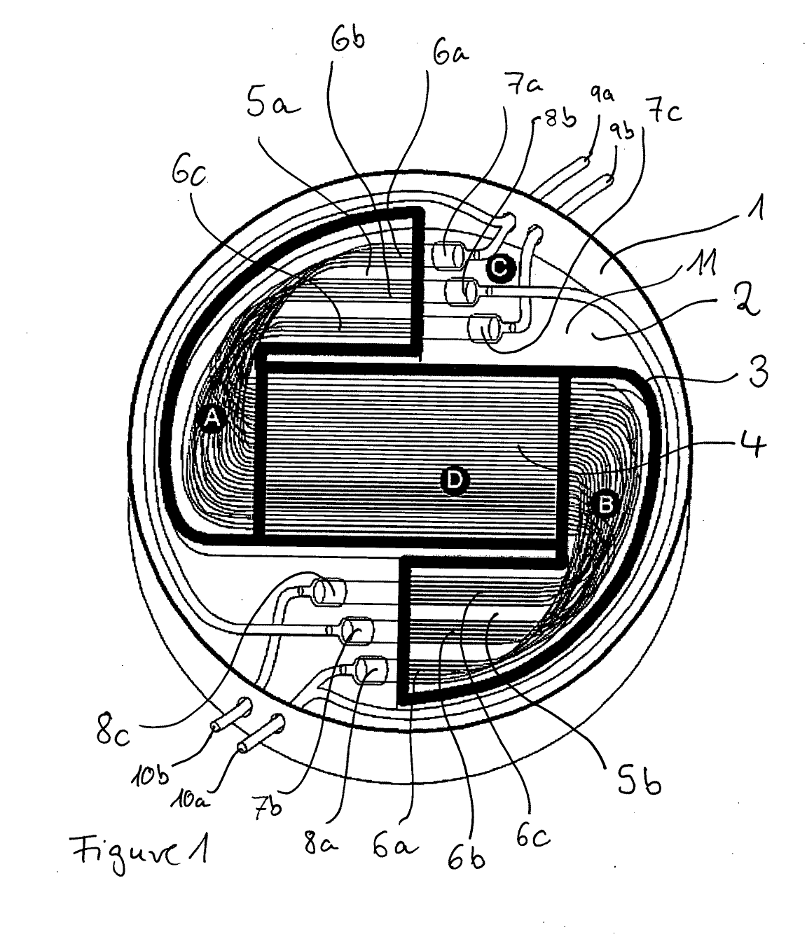

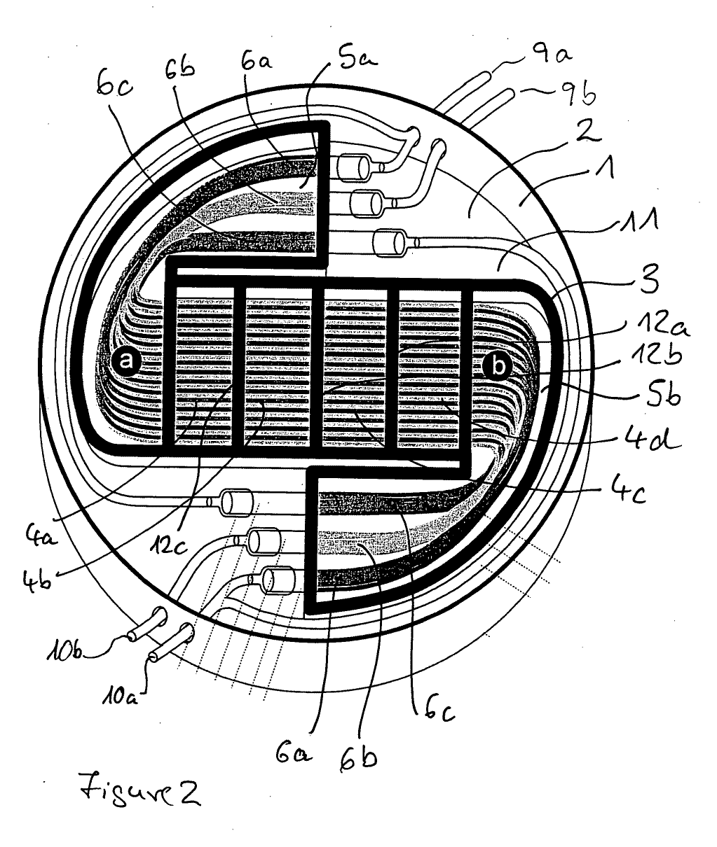

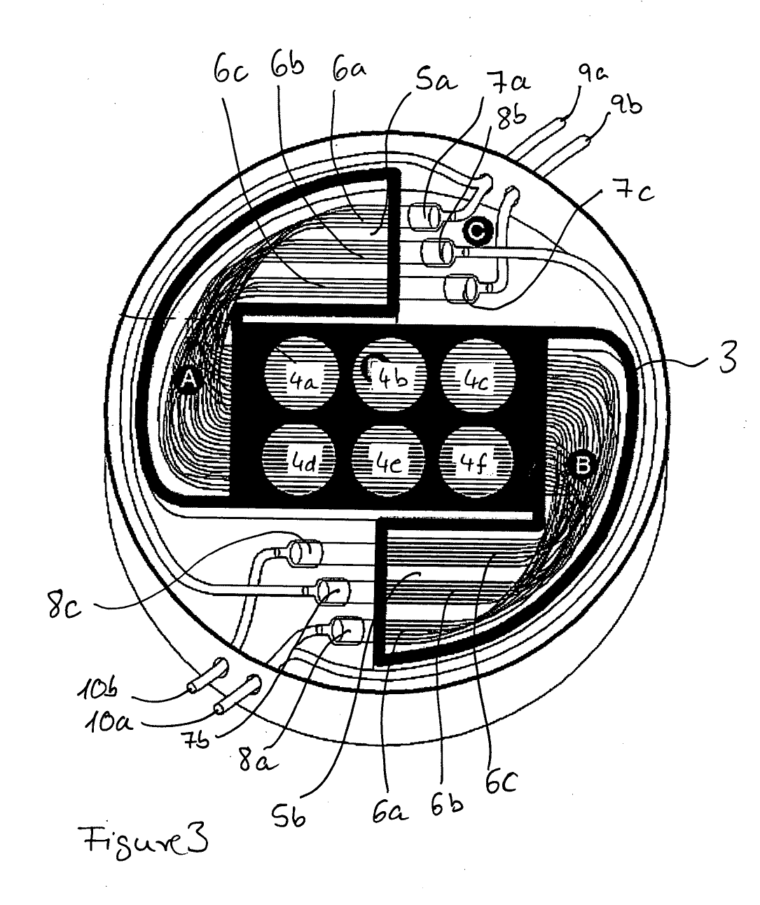

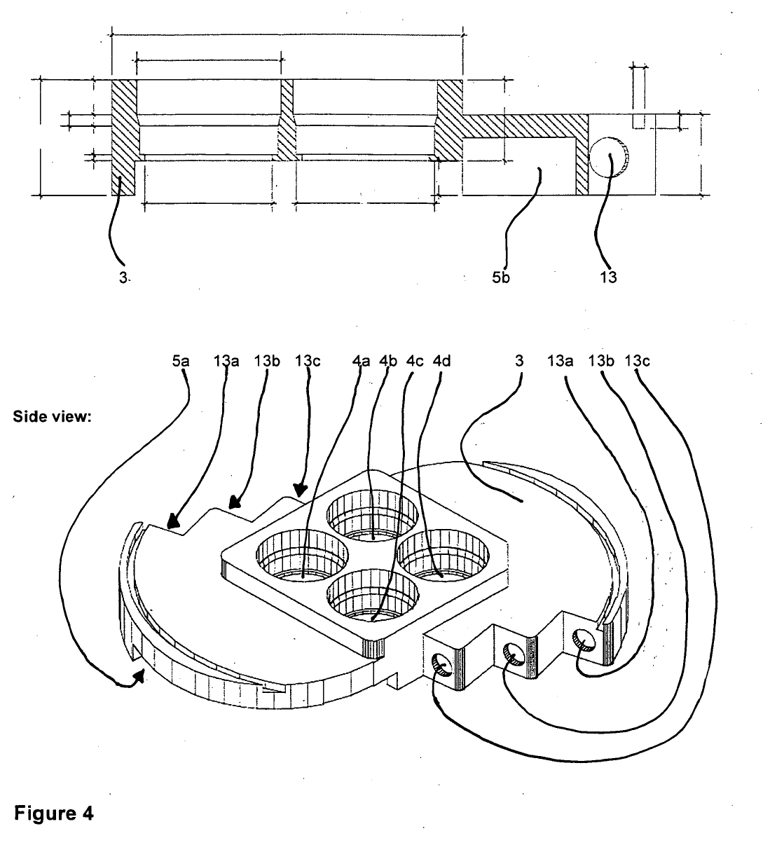

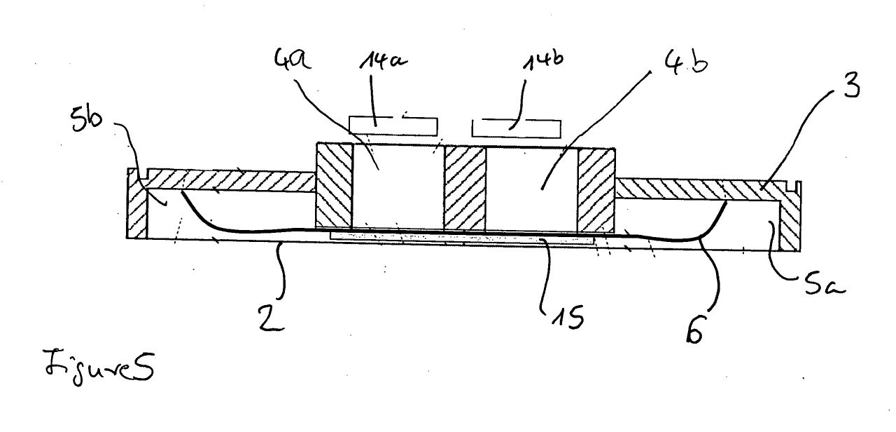

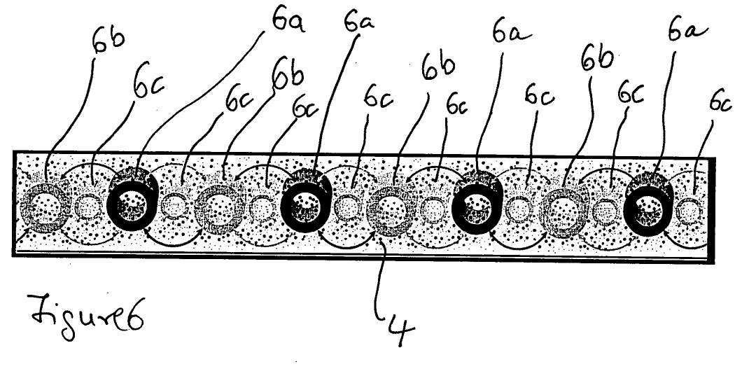

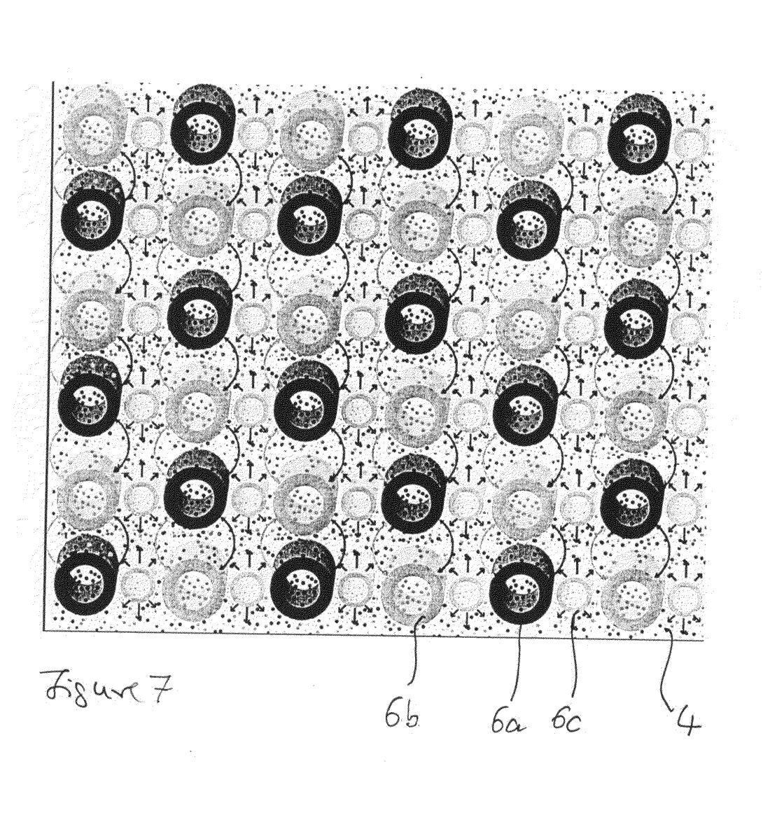

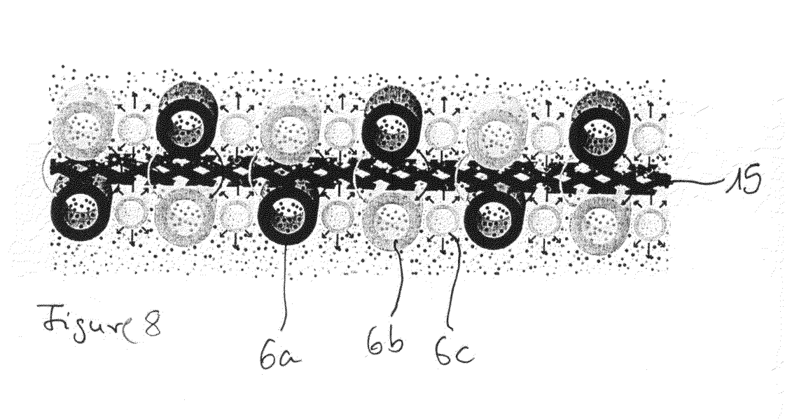

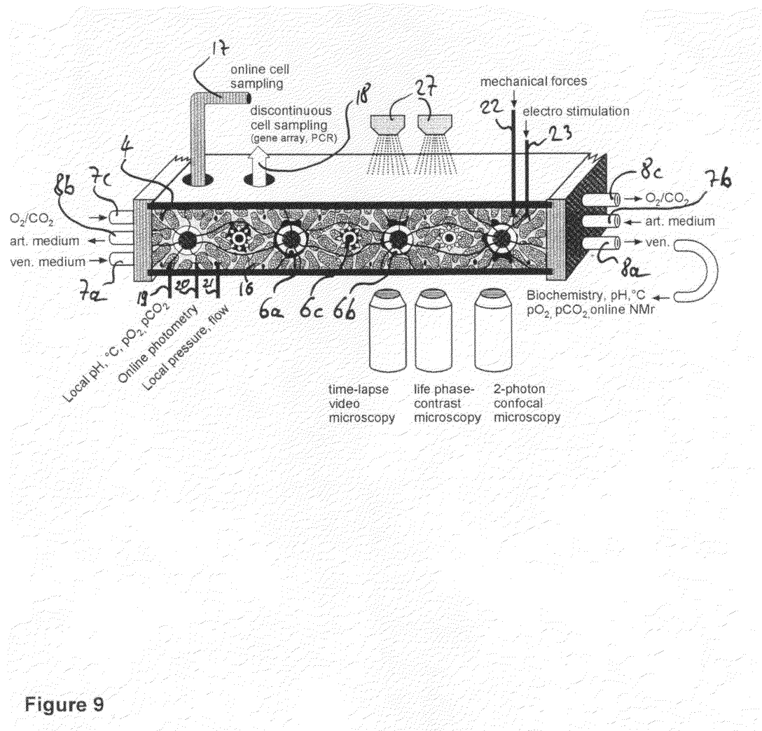

FIG. 2 shows a modified Petri-dish according to FIG. 1. It exhibits a block 3 that consists of 6 spaces 5a, 5b, 4a, 4b, 4c, 4d. The spaces 4a, 4b, 4c, 4d are used as culture spaces, while the hollow fiber capillaries of the hollow fiber capillary systems 6a, 6b, 6c are bundled in the curved spaces 5a and 5b and fixated, stabilized and sealed by casting the spaces 5a and 5b with plastics. In the culture spaces 4a, 4b, 4c, 4d the hollow fiber membranes of the hollow fiber membrane systems are in arranged in parallel. In the areas 5a and 5b they run along an 180°-curve. Within the areas 4a, 4b, 4c, and 4d, the hollow fiber membranes of the hollow fiber membrane systems 6a, 6b, 6c are interwoven in such a way that one hollow fiber membrane of system 6a is located next to a hollow fiber membrane of system 6b which, in turn, is located next to a hollow fiber membrane of system 6c followed by a hollow fiber membrane of system 6a, and so on and so forth.

The areas 5a and 5b of block 3, that are cast with plastics, are bent in such a way that they follow the 180°-curve of the hollow fiber membranes.

In this example, the hollow fiber membrane system 6b is supplied independently from hollow fiber membrane systems 6a and 6c via inlet 9b and outlet 10b, while hollow fiber membrane systems 6a and 6c are supplied in counter current flow. The dividing walls 12a, 12b, and/or 12c between the culture spaces 4a, 4b, 4c, 4d can, according to the invention, feature or consist of flat membranes or biomaterial.

The four culture spaces 4a, 4b, 4c, 4d are separate from each other, so that cells cannot escape from either one, but medium and cell product/mediator exchange can occur between all culture spaces in case semi-permeable hollow fiber capillary membranes are used.

Also, according to the invention, several culture spaces 4a through 4d can be closed with a lid or the entire culture dish can be closed with a lid.

FIG. 3: A modified culture dish according to the invention, with three hollow fiber membrane systems and six circular culture spaces.

FIG. 3 shows a modified Petri-dish according to FIGS. 1 and 2, but with six circular culture spaces. The culture spaces are enabled in the same way as in FIG. 2, however, the culture space is further divided into more distinct culture compartments in which the cells can be separated but the perfusate can communicate via the hollow fiber membrane walls or the membrane walls between the compartments; this way six separate cell populations can be cultured in compartmentalized co-culture but they can communicate via soluble medium factors/mediators. Contrary to FIG. 1 that only consists of one culture space 4, here block 3 separates six circular culture spaces 4a, 4b, 4c, 4d, 4e, and 4f. These culture spaces are arranged in such a way that three culture spaces 4a, 4b, 4c respectively 4d, 4e, 4f are arranged one after another along the running direction of the hollow fiber capillaries leading each hollow fiber membrane through three culture spaces. According to the invention, vertical connections can be installed between the side-by-side arranged culture spaces located in the running direction of the hollow fiber membranes. It is possible to connect culture space 4a with culture space 4d and/or culture space 4b with culture space 4e and/or culture space 4c with culture space 4f with further capillary membranes. This is of interest for neuronal cells, which send neurites through macroporous walls of suitable capillaries from one culture space to the next. In the example at hand, a substance, e.g. gas, can flow through the capillary membrane system 6c independently from the capillary systems 6a and 6b that passes substances in counter current flow. According to the invention, several culture spaces 4a through 4f can be closed with a lid or the entire culture dish can be closed with a lid. This lid can prevent medium shift during high flow/pressure operation. If single lids are used to close a culture space 4a through 4f, the lid can be equipped with a vent for easy opening and closing without generating pressure changes in the culture space. Consequently, venting allows insertion/removal of the lid without pressure generation in the cell compartment and without medium shift during insertion/removal.

This vent can additionally be used for cell injection to fill the culture space with cells; it can also be used to take samples, or to insert probes to measure, e.g. temperature or pH-value.

FIG. 4: A core to produce a mold for the manufacturing of the modified culture dish according to the invention.

FIG. 4 shows a perspective view of a core to produce a mold to manufacture the inner block of the modified Petri-dish. This refers to the block 3 of the inner space 11 in FIGS. 1 to 3.

As a result of using the core, producing a mold and manufacturing the inner block with that mold, a structure for the casting of capillary membranes with three hollow fiber membrane systems is prepared. This facilitates the production of the modified culture dish. This structure is filled with, e.g. capillary membranes which, consequently, can be easily cast. At the end, this structure with the capillaries can be inserted or glued into a conventional Petri-dish.

This example of a core, which leads to a culture dish with four circular culture spaces exhibits guides for the fixation of the hollow fiber membranes systems.

As described afore, the areas 5a and 5b are cast with plastics after the hollow fiber capillary membranes have been placed.

Block 3 also exhibits guides 13a, 13b and 13c for the combined inlets or outlets 7a, 7b, 7c or 9a, 9b, as well as guides 14a, 14b, 14c for the combined inlets or outlets 8a, 8b, 8c or 10a, 10b.

FIG. 5: A section of a modified culture dish according to the invention, through the central block/core according to FIG. 4. It indicates how a cast block is used to assemble the capillary membranes and upper lids or lower bottoms as an insert into a conventional Petri-dish. The production of a two-culture space modified culture dish for two independent 3D cell spaces is illustrated.

FIG. 5 shows a section through the assembled block 3 according to FIG. 4, which can be inserted into a standard Petri-dish. The areas 5a, and 5b are cast with plastics after the hollow fiber capillary membranes 6 have been placed. FIG. 5 shows an example in which hollow fiber capillary membranes only pass through the lower area of the culture spaces 4a and 4b. Here, hollow fiber capillary membranes 6 are arranged in one plane.

FIG. 5 easily explains the assembly process of the culture dish according to the invention. First, at the contact points, the hollow fiber membranes 6 are glued to the base of the molding 3 and the bottom side of block 3. The ends of the hollow fiber membranes are fed through the connection points 7a, 7b, 7c und 8a, 8b, 8c. To seal the culture space 4a and 4b a glass plate 15 or a flat membrane is glued on. Subsequently, the spaces 5a and 5b are cast with plastics to seal all capillary membranes. Now the molding can be inserted into a standard Petri-dish. As necessary, culture spaces 4a and 4b can be closed with lids 14a and 14b.

FIG. 6: A section of a device according to the invention, through an arrangement of individual hollow fiber membranes for a culture dish with three hollow fiber membrane systems arranged in one plane.

FIG. 6 shows a section trough a hollow fiber capillary membrane system as existent in culture spaces 4, in FIG. 1-3 and 5. The slice runs perpendicular to the hollow fiber capillaries. This example also exhibits hollow fiber membrane systems 6a, 6b und 6c. Medium, liquid and/or other substances can be supplied or discharged through 6a and 6b. Gas, e.g. oxygen, is supplied via the hollow fiber membranes 6c for oxygenation, which is release into the medium via the membranes. Here, the hollow fiber membranes are arranged in one plane in such a way that the hollow fiber membranes 6a and 6b alternate with each one hollow fiber membrane 6c between them, thus enabling the highest equability of substance distribution in one plane.

FIG. 7: An illustration of the scale up of the membrane configuration in culture space 4 the modified culture dish, from one layer (see FIG. 1), to six layers.

FIG. 7 shows how further layers can be incorporated into the modified Petri-dish; and thus the scale up of the cell number to be studied or to be removed after culture. Scale up of the membranes in the dish, from one layer (FIG. 1), to two layers (FIG. 8), to further layers enables a scale up of the cell number to be cultured for research, cell differentiation, cell production, industrial, or therapeutic use.

FIG. 7 shows a section through an arrangement of hollow fiber capillary membranes 6a, 6b, 6c in a culture space 4, whereby several of the arrangements described in FIG. 6 are arranged in parallel on several planes. A hollow fiber capillary membrane 6a is arranged next to a hollow fiber capillary membrane 6b with a hollow fiber capillary membrane 6b placed in between each. The planes are stacked in such a way that, also in the vertical direction to the base plate 2 of the modified Petri-dish, a hollow fiber capillary membrane 6a is arranged next to a hollow fiber capillary membrane 6b in an alternating pattern. This configuration enables even distribution of medium, fluids and/or other substances, as well as gases, throughout the entire culture space. Thus, a larger cell mass can be supplied, where gradients and mass exchange properties are comparable to the smallest scale.

FIG. 8: A section through another arrangement of a modified culture Petri-dish according to the invention. It describes a double layer membrane configuration, where a biomaterial for testing cell-biomaterial interactions is interposed between the hollow fibers. This modification allows the use of the invention for the testing of biomaterials or for testing cell-biomaterial interactions.

FIG. 8 shows three hollow fiber membrane systems, 6a, 6b, and 6c that are arranged in one plane as in FIG. 6, however, two of these planes are arranged in parallel and separated by a layer of a biomaterial 15. As a result, two layers of hollow fiber membranes with an inlay of biomaterial can be offered to test the cells-material interaction. Thus, the invention can be utilized to test materials or cell-material interactions.

FIG. 9: A section through a modified culture dish according to the invention, with additional features and/or devices to measure and control the medium properties and the cell behavior.

FIG. 9 shows possible advantageous further modifications or embodiments of the invention. It describes the use of the invention for studying cell behavior with life microscopy methods and additional probes/sensors.

Once again, a cell culture 16 is presented in a culture space 4, which is filled with hollow fiber membranes 6a, 6b, 6c. Here, the hollow fiber membranes serve to supply and/or remove substances and/or gasses. For this, for example, medium can be transported via inlet 7a. The hollow fiber membranes 6b, serve to remove substances via outlet 7b. Within the cell culture 16, arrows 27 indicate the substance flow direction. Moreover, the hollow fiber capillary membrane system 6c serves for gas supply and -removal with oxygen and carbon dioxide via inlet 7c or outlet 8c.

Cells can continuously be removed from the cell culture via connection 17 (Online-cell-sampling). Cells can be removed discontinuously via opening 18, e.g. gene arrays or polymerase-chain reaction tests. This can alternatively be done through lid 14 in FIG. 5. Additionally, the culture dish exhibits transducers 19, 20, and 21 that can measure, e.g. the local pH-value, temperature and/or pressure of oxygen and/or carbon dioxide, extinction, fluorescence or other parameters. This, online photometry can be performed as well as pressure and flow can be determined.

Mechanical force can be applied or electro stimulation can be performed to the cell material via connections 22 and 23.

According to the invention, the Petri-dish can be designed in such a way that time-lapse video microscopy, live phase contrast microscopy and/or confocal 2-photon-microscopy can be performed via microscope 24, 25 and/or 26. The venous medium is removed via outlet 8b and can be utilized for online biochemical studies with suitable devices to measure pH, temperature, oxygen and/or carbon dioxide pressure, fluorescence, as well as NMR (Nuclear Magnetic Resonance) studies. According to the invention, a modified culture Petri-dish would be one that exhibits only parts of the described features and/or devices for the analysis and manipulation of cell cultures described in this example.

### DESCRIPTION OF THE INVENTION

The function of the invention at hand is to offer a device in which cells can be cultivated in an environment that is similar to the natural cell environment, but is also accessible for imaging and cell sample removal during culture, and facilitates broad studies, including imaging, of cell development and cell culture as a whole. This task is solved with the culture dish according to claim 1. Advantageous further developments of the culture dish, according to the invention, are further explained in the claims section.

According to the invention, a hollow fiber membrane system with a multitude of hollow fiber membranes that, on one end, are bundled into a joint inlet and on the other end into a joint outlet, are arranged in a culture dish or dish that exhibits a base plate and a rim along the base plate. Fluids, media, gas and/or other substances can be passed through the hollow fiber membranes of at least one hollow fiber membrane system. For instance, the Petri-dish can be filled with cell culture that is supplied with nutrients via the hollow fiber membranes. Such a cell culture can also be supply with oxygen or other gases. At the same time, one or multiple hollow fiber membrane systems can serve for the removal of metabolic waste.

In latter case, the two-component system (with a cell culture compartment and a second compartment in the lumen of the first hollow fiber membrane system) becomes a multi-compartment system (with further compartments in the lumen of the further hollow fiber membrane systems).

The hollow fiber membranes are preferably developed tube-like or channel-like. Hydrophylic micro-filtration membranes are especially suitable for the perfusion of media through the content of the culture dish. Hydrophobic oxygenation capillary membranes are preferred to supply oxygen and/or other gases. The hollow fiber membranes of one hollow fiber membrane system are arranged in such a way that the cultivating medium can be supplied most evenly and substances can be removed most evenly. For instance, hollow fiber membranes of one hollow fiber membrane system can run alternating to each other with. the hollow fiber membranes of a second hollow fiber membrane system, and both can be operated in counter current flow operation.

In a preferred embodiment, the culture spaces exhibit an inner diameter von 5-100 mm, preferably 10-30 mm.

In an advantageous design of the invention at hand, the interior space of the dish (the cell compartment) is divided into two or more distinct cell spaces within the dish. The interior space of all culture spaces, consists of the area contained by the base plate and the rims surrounding it. In a culture dish with several distinct culture spaces, several different cultures can be cultivated simultaneously in separate culture spaces but in one device, where the cells are separated but the medium with its factors and mediators can flow through all culture spaces. If the culture spaces are arranged in parallel to the base plate, they are easily accessible from the top. Here, the hollow fiber membranes can be arranged in such a way that each hollow fiber membrane first passes through one culture space, then through the next, and so on so forth, thereby passing through two or more culture spaces consecutively. Thus, each hollow fiber membrane can supply several or all culture spaces. With this arrangement it is possible to pass products generated in one culture space to another culture space via the hollow fiber membranes, thus enabling substance exchange between the individual culture spaces. This substance exchange can be applied to mediators, effectors or antibodies, metabolic substances, differentiation factors, growth factors and such. Substance exchange can also occur as gas exchange. One or several hollow fiber membrane systems can be arranged in such a way that they connect individual culture spaces but do not have an outside connection, thereby circulating substances within the culture spaces in a closed system. It is also possible that hollow fiber membranes of one hollow fiber membrane system pass through one culture space and subsequently split up into different culture spaces. The culture spaces can be supplied one after another or in parallel.

The culture spaces can have any shape, e.g. round, square, hexagonal, etc. If two culture spaces are arranged next to each other and share a wall, this wall can exhibit or consist of a flat semipermeable membrane. Certain substances can pass through this flat membrane between the culture spaces without having to go through the hollow fiber membrane systems.

According to the invention, the culture space and/or the walls can exhibit biomaterial or the walls can consist of biomaterial, thus enabling to analyze the cell culture, e.g. the interaction of the biomaterial with the cells of the cell culture and vice versa.

According to the invention, two or more culture spaces can be arranged on top of each other in perpendicular direction to the base plate. In this case, hollow fiber membranes of one or several hollow fiber membrane systems can pass through the bottom of one culture space upward or downward into the above or below culture space. The wall between the culture spaces can exhibit or consist of a membrane and/or biomaterial to enable even perfusion of substances into several or all culture spaces. For Example, a hydrophobic or oxygen permeable membrane enables additional oxygenation. According to the invention, a shared wall between two culture spaces can also exhibit biomaterial through which the interaction between the cells and the biomaterial can be tested.

The hollow fiber membranes of one or several hollow fiber membrane systems can pass through one or several culture spaces in different ways. The actual arrangement depends on the concrete application. It is particularly advantageous if the hollow fiber membranes of one or several hollow fiber membrane systems are arranged in parallel to each other, whereby the hollow fiber membranes of different hollow fiber membrane systems can be arranged in one plane or in different planes. The hollow fiber membranes of one hollow fiber membrane systems can run in parallel to each other and intersect the hollow fiber membranes of another hollow fiber membrane system at an angle. In this case it is particularly advantageous if the hollow fiber membranes of two or three hollow fiber membrane systems are interwoven with each other. Such an arrangement enables even perfusion of media and the study of cell-cell-interactions at high tissue densities. Here, a physiological microenvironment of the cells can be achieved, including homeostasis of oxygen, electrolytes, nutrients, soluble factors, and analogous pH-value while avoiding metabolic gradients. Depending on the task at hand, these advantages can also be achieved by applying other hollow fiber membrane arrangements.

In another particularly advantageous design of the invention at hand, the hollow fiber membranes of two or several hollow fiber membranes systems are arranged in such a way inside the culture space that enables the perfusion with culture media in counter current flow. Thus, media can flow through a number of hollow fiber membranes in one direction, and through another set of hollow fiber membranes, preferably arranged in parallel, in opposite direction. It is also possible that a substance flows through one hollow fiber membrane systems in one direction and another substance flows through another hollow fiber membrane system in the opposite direction, thereby enabling even substance exchange with the culture space and the cells.

A further design of the culture dish consists of an arrangement of several culture spaces that are interconnected through individual hollow fibers. If these hollow fiber membranes exhibit small pores, e.g. 1-5 micrometer in size, cell projections, e.g. neuronal cell projections (neuritis) can pass through them from one culture space to the next, yet leaving the cell behind in the culture space.

According to the invention, the network of capillary membranes can be tightly packed or consist of several alternating layers, whereby each layer is an independent system. The first layer, consisting of individual hollow fiber membranes is arranged in parallel to the base plate. The second layer, also consisting of individual hollow fibers is arranged either on the same plane, or on a parallel plane, or opposite the first layer, e.g. rotated at a 90° angle. Analogous, other layers can be arranged at other angles. Biomaterial can be placed between these layers for cell analysis.

In all arrangements of the invention, polypropylene, polyamide, polysulfon and/or cellulose, or silicon rubber is the preferred material for hollow fiber membranes. The selection of the hollow fiber membranes depends on the molecules and/or cells chosen for the mass transfer. However, any existing state-of-the art hollow fiber membranes, known for mass transfer can be used. In all cases, the hollow fiber membranes can be developed as tubes, channels, and/or capillaries.

In another advantageous arrangement, fluid-impermeable hollow fibers and/or capillaries, that transfer tempered fluids, pass through one or several culture spaces, enabling exact temperature setting of the cultivating fluid or culture media. Here, the fluid is tempered in an external vessel outside the culture space.

In another embodiment of the invention, the cell compartment between the hollow fiber membrane systems is filled with a mixture of cells and gels, e.g. made of collagen, to improve cell or cell aggregate immobilization in a 3-D arrangement between the membranes.

In another arrangement of the invention, the hollow fiber membrane systems are treated with a surfaces coating, e.g. collagen, fibronectin or laminin to improve cell adhesion or cell aggregate immobilization between the membranes.

It is particularly advantageous if the culture dish and/or at least one of the culture spaces can be closed with a lid, thus enabling high flow rates without pressure build-up, and preventing fluid displacements inside the Petri-dish. Openings in the lid that can be closed with e.g. a Luer-lock connection facilitate easy closing of the interior space without pressure- or fluid shifts. Air- and fluid displacements can be released through theses openings. Measuring devices to measure media properties, e.g. pressure, pH-value and/or temperature can also be connected to the lid, but also through any part of the housing. It is also possible to insert cells, microorganisms, or other substances into the culture dish through these opening, as well as take samples, or vent the chamber, which for example enables easy removal of the lid.

In another advantageous arrangement one or several culture spaces are arranged in one connected block. This block can exhibit channels for hollow fibers, and/or one or several hollow fiber membrane systems, and/or inlets, and/or outlets. Fixtures to attach measuring instruments, lids, etc. can also be installed on this block. Such a block enables the utilization of standard Petri-dishes for cell culture that are simply inserted into the connected block. The block can also consist of multiple parts. It can be arranged in such a way that it enables quick and easy exchange of hollow fibers or flat membranes. For instance, it can consist of a bottom part onto which the hollow fiber membranes of one or several hollow fiber membrane systems are placed.

Subsequently, the top part of the block that is partitioned into several culture spaces is placed on top. The bottom part can exhibit dents at a non-evanescent angle to the base plate for which the top part exhibits the corresponding omissions, thereby enabling the arrangement of hollow fiber membranes of one or several hollow fiber membrane systems on planes that are not parallel to the base plate.

A particular advantage of the modified Petri-dish at hand is that it enables detailed analysis and studies of the content in the culture dish without having to disturb the culture medium by exhibiting an imaging “window” in the base plate, in the rim, and/or in one or several lids through which microscopic analysis is possible. An easy embodiment is to produce the bottom of the cell compartment with microscopy glass slides and the lids out of imaging quality glass or plexiglass. Thus it is possible to use, e.g., time-lapse video microscopy, phase-contrast microscopy, or confocal 2-photon microscopy. The interior space of the culture dish can be illuminated through windows located on the opposite side of the microscopy window. This function can also be achieved through transparent lids above the culture space.

An advantageous arrangement of the invention at hand is devices that are installed on the culture dish and/or the block to measure properties of the content inside the dish, e.g. the pH-value in a certain location, the temperature, as well as O₂ and/or CO₂ pressure. Local pressure- and flow measurements are possible as well as online photometry or fluorescence or NMR studies. The invention at hand can also exhibit devices that produce mechanical force, electro-stimulation or build up electrical fields.

Another advantage of the invention at hand is that in another embodiment the entire construction, or parts of the housing, can be build out of flexible materials, so that mechanical forces or movements can be applied via the housing to the cell compartment; leading to movements of the cells. This can be advantageous for cell production based on enzymatic passaging, since enzymatic passaging can be improved by additional cell movements.

According to the invention, the culture dishes cannot only be utilized individually but several culture dishes can be connected to each other, enabling fluids, mediators, factors, gases and/or other substances to pass through at least two culture spaces consecutively or at the same time. Thus several “organ systems” with organ specific cells can be used in one circuit, where either the medium lines allow soluble factor exchange or the cell compartment openings can be connected allowing cell migration between “organ system” like culture dishes. Such a configuration allows stem cell stimulation from other cell systems, while they reside in their “organ typical anatomic niche”. It also allows cell maturation via cell migration from one “organ system to the next”, of interest, e.g. for immune cell maturation or sensitization and blood cell maturation or production.

The invention at hand has, compared to state of the art technology, many advantages:
With the invention at hand, high tissue density 3-D culture can be achieved as it is present in the natural cell environment
The culture can also be perfused in a 3-D space at high cell densities.
Decentralized mass exchange can be performed eliminating gradients
Integral gassing, including oxygen supply and carbon dioxide removal, is possible
The cell culture is accessible for analysis through cell sample taking during culture at any time point
The cells can be imaged through microscopic methods on-line without affecting or disrupting them
Shared walls that have been equipped with membranes and/or biomaterial can be setup between individual shared culture spaces enabling the analysis of cell-material interaction with e.g. biomaterial, material can also be interposed between two membrane layer
The fabrication of a mold made of a single block enables the manufacturing and utilization of this invention with standard Petri-dishes. Thus, precise studies of cell growth are possible in standard culture dish fixtures, e.g. under the microscope.
Because each individual hollow fiber membrane can pass through several culture spaces, selected and controlled substance redistribution of substances originating in one culture space to the other culture spaces is possible. The concentration of individual substances can be manipulated by varying the density of the hollow fiber membranes. Variations in the capillary membrane wall permeability can influence substance exchange with respect to molecule size. This allows the generation of several organ-typical cell cultures (one in each cell compartment) in the same modified culture dish, while soluble factor exchange can be enabled in a controlled manner.

### EXAMPLE

A production process for one embodiment of a modified Petri-dish according to the invention was developed. This example is illustrated in FIGS. 4, 5 and 6. The example leads to a culture dish with four circular culture spaces. This example compares to FIG. 3, except that the modified culture dish in FIG. 3 represents six culture spaces. The outer diameter of the produced embodiment is 140 mm, the height is 20 mm; each of the resulting four culture spaces exhibit an inner diameter of 25 mm.

FIG. 4 shows a perspective view of the core used to produce a mold for manufacturing the inner block of one embodiment of the modified culture dish. As a result of using that core, producing a mold and manufacturing the inner block with that mold, a structure for the casting of capillary membranes with three hollow fiber membrane systems was prepared. This facilitated production of the modified culture dish. This structure was filled with oxygenation hollow fiber membranes (Oxy+, Membrana, Germany) and filtration capillary membranes (Plasmaphan, Membrana, Germany) which consequently could easily be cast with two component polyurethane (PUR, Morton, Germany). At the end, this structure with the capillaries was inserted and glued into a conventional Petri-dish.

FIG. 4 shows the core, which exhibits guides for fixing the hollow fiber membranes systems.

The areas 5a and 5b are cast with polyurethane after the hollow fiber capillary membranes have been placed.

Block 3 also exhibits guides 13a, 13b and 13c for the combined inlets or outlets 7a, 7b, 7c or 9a, 9b, as well as guides 14a, 14b, 14c for the combined inlets or outlets 8a, 8b, 8c or 10a, 10b.

In FIG. 5 a section of the resulting modified culture dish is described. After a block was produced (through the core according to FIG. 4), this block was used to assemble the capillary membranes and upper lids and lower bottoms as an insert into a conventional Petri-dish.

In this example three capillary membrane systems were used, two hydrophilic for medium perfusion in counter current flow operation and one hydrophobic for oxygenation; each hydrophilic membrane system exhibited 12 single hollow fiber membrane capillaries; the hydrophobic system 24. All capillaries were assembled with a distance of 0.8 mm to each other. The capillaries were all arranged in one plane, in an alternating pattern so that the capillaries from all three systems were equally distributed in that plane. Four cell culture spaces were laid over these capillaries, by assembling two cell spaces laid over one half of these capillaries, so that each cell space contained parts of 24 capillaries, and two of the cell culture spaces shared the same capillaries in different areas. The production resulted in four-culture space modified culture dish for four independent 3D perfuseable cell spaces.

FIG. 5 shows a section through the assembled block 3 according to FIG. 4, which was inserted into a standard Petri-dish. The areas 5a, and 5b were cast with polyurethane after the hollow fiber capillary membranes 6 have were placed. In this embodiment, all hollow fiber capillary membranes 6 are arranged in one plane.

FIG. 5 explains the assembly process of one embodiment of a culture dish according to the invention. First, at the contact points, the hollow fiber membranes 6 were glued with polyurethane to the base of the molding 3 and the bottom side of block 3. The ends of the hollow fiber membranes were fed through the connection points 7a, 7b, 7c und 8a, 8b, 8c. To seal the culture space 4a and 4b a microscopy glass plate 15 was glued on. Subsequently, the spaces 5a and 5b were cast with polyurethane to seal all capillary membranes. Now the molding was inserted into a standard Petri-dish. The culture spaces 4a and 4b were closed with lids 14a and 14b, lathed out of transparent acrylic. Holes were drilled into these lids and Luer-lock connectors for venting and cell inoculation were glued into these lids.

FIG. 5 shows a cross view though the cell compartment between the acrylic lid (upper face) and bottom microscopy glass slide.

In using the produced devices, cells were inoculated into the cell culture spaces together with culture medium, and phase contrast microscopy was performed. In one example of using the device, the cells were inoculated into the cell space, and between the hollow fiber capillary membranes after the cell suspension was mixed with collagen, where the collagen solidified after inoculation, keeping the cells within a gel in between the hollow fibers.

Modifications and variations of the described methods and device of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the relevant field in which this invention resides, are intended to be within the scope of the described claims.

1. A modified culture dish with a base plate and a rim along the base plate that, together with the base plate, delineates the interior space of the culture dish against the outer space with a wall that is open on the top and can be closed by a lid for allowing cell sample removal, including a transparent lid for allowing imaging and, thereby characterized
that in the interior culture space at least two or more, preferably three to four and most preferably three, independent hollow fiber capillary membrane systems, each exhibiting a multitude of hollow fiber membranes, that each on one end are bundled into a joint inlet/manifold and on the other end into a joint outlet/manifold, through which fluids/media and/or gas are passed from the outer space via the inlet(s) and outlet(s),
that the hollow fiber membranes of at least one hollow fiber membrane systems are semi permeable hydrophobic, including oxygenation capillary membranes, and/or hydrophilic, including microfiltration capillary membranes,
that the hollow fiber membranes are made of, or contain, polypropylene, polyamid, polysulfon, polystyren, polystyrol, cellulose and/or silicon rubber, cell culture grade or medical grade polymers,
that the capillary membranes of at least one membrane system exhibit an outer diameter of 200-1000 micrometer, preferably between 400-800 micrometer, and a wall thickness of 20-200, preferably 30-100 micrometer,
that media, fluid or gas, pass through the hollow fiber membranes of at least two hollow fiber membrane systems,
that these capillary bundles and in-/outlets are embedded in said wall.
that the capillary hollow fiber membranes are arranged in an alternating pattern in the culture space, whereas the hollow fiber membranes of at least one of no less than two hollow fiber membrane systems are—at least in sections—arranged in parallel to each other in one plane, whereas the capillaries of at least two of the hollow fiber membrane systems are arranged alternatingly,
that the culture dish exhibits at least one inlet, preferably exhibiting a Luer-lock or similar connection, through which microorganisms can be imported, and/or venting is possible, through which devices to measure pressure, pH-value, extinction, fluorescence, and/or temperature can be attached.

2. A culture dish, according to afore-mentioned claim, thereby characterized that the interior space is divided into at least two or more, preferably six, and most preferably four culture spaces, whereas
the hollow fiber membranes of at least one of no less than two hollow fiber membrane systems can pass through either one or more of the at least two culture spaces,
at least one of the at least one culture spaces is square-, round- or oval shaped,
at least two of no less than two culture spaces are arranged side by side and in parallel to the base plate, or whereas at least two of no less than two culture spaces are arranged on top of each other and perpendicular to the base plate.

3. (canceled)

4. (canceled)

5. A culture dish, according to at least one of the afore-mentioned claims, thereby characterized that at least two of no less than two culture spaces are separated by a shared wall, thereby characterized that the shared wall can exhibit a membrane, a semi-permeable membrane, and a material, e.g. a biomaterial, for material-cell interaction analysis.

6. (canceled)

7. (canceled)

8. (canceled)

9. (canceled)

10. (canceled)

11. A culture dish according to at least one of the afore-mentioned claims, thereby characterized that the hollow fiber membranes of the first hollow fiber membrane system run in parallel to each other—at least in sections—and in parallel and alternatingly to the hollow fiber membranes of the second or further hollow fiber membrane systems, whereas the hollow fiber membranes of the one and second or further hollow fiber membrane systems are all arranged in parallel and a) alternatingly all in one plane, or b) are arranged in parallel whereas the hollow fiber membranes of the one and the further hollow fiber membrane systems are each arranged in one plane but each system on different planes and whereas the hollow fiber membranes of the first system cross over the hollow fiber membranes of the second or further hollow fiber membranes systems (and thus cross each other e.g. in an angle of 90 degrees), or c) whereas the hollow fiber membranes of the first hollow fiber membrane system are interwoven with the hollow fiber membranes of the second or further hollow fiber membranes systems at least in sections, or combinations of a)-c).

12. (canceled)

13. (canceled)

14. (canceled)

15. A culture dish according to at least one of the five afore-mentioned claims, thereby characterized that a first perfuseable and with a circuit connectable and a second non connected but perforated hollow fiber membrane system is arranged inside the interior space of the culture dish.

16. (canceled)

17. (canceled)

18. (canceled)

19. (canceled)

20. (canceled)

21. (canceled)

22. (canceled)

23. (canceled)

24. A culture dish according to at least one of the afore-mentioned claims, thereby characterized that that the hollow fiber membranes of at least two hollow fiber membrane systems are supplied with media via a shared inlet line and a shared outlet line whereas at least two of the hollow fiber membranes systems are arranged in such a way that media can pass through in a counter-current/counter directional flow pattern involving the lumen of the at least two capillary systems and the cell compartment.

25. (canceled)

26. (canceled)

27. (canceled)

28. A culture dish according to at least one of the afore-mentioned claims, thereby characterized that tempered liquids or gas can be passed through the internal space of the culture dish via fluid/substance impermeable hollow fibers, whereas the hollow fiber of at least one hollow fiber system is used for tempering of the culture space and/or for heat exchange for cryopreservation of cells in the culture space.

29. A culture dish according to at least one of the afore-mentioned claims, thereby characterized that the base plate exhibits at least one flat membrane that is oxygen permeable and/or hydrophobic.

30. (canceled)

31. (canceled)

32. (canceled)

33. A culture dish according to at least one of the afore-mentioned claims, thereby characterized that the hollow fiber membranes of at least one hollow fiber membrane system, and/or any other surface/membrane in the culture space, exhibit a surface coating, e.g. made of collagen, for improving cell adhesion and/or enabling call aggregate immobilization, and/or whereas at least one of the at least one cell compartments is filled with a mixture of cells and micro carriers/spheres, and/or gels, e.g. collagen containing gel.

34. (canceled)

35. A culture dish according to at least one of the afore-mentioned claims, thereby characterized that the interior space of the culture dish and/or at least one culture space can be closed with a removable lid allowing cell sampling, or that the upper part of the dish is closed with a sealed upper part, whereas this lid or upper part can exhibit at least one inlet, preferably exhibiting a Luer-lock or similar connection, through which microorganisms can be imported, and/or venting is possible, through which devices to measure pressure, pH-value, extinction, fluorescence, and/or temperature can be attached.

36. (canceled)

37. (canceled)

38. A culture dish according to at least one of the afore-mentioned claims, thereby characterized that several culture spaces are arranged in a connected block of which at least one culture space and/or channels, and/or hollow fiber membranes of at least one hollow fiber membrane system, are housed for supply and removal.

39. A culture dish according to at least one of the afore-mentioned claims, thereby characterized that the base plate exhibits at least one window through which the content of the culture space can be viewed through a microscope, and/or that culture space exhibits at least two opposite walls/membranes/windows or lids, e.g. made out of microscopy grade glass or transparent culture grade plastics.

40. A culture dish according to at least one of the afore-mentioned claims, thereby characterized that the culture dish exhibits sensors and/or effectors to study cell behavior and/or measure the pH-value, extinction, pressure, fluorescence, temperature and/or other parameters.

41. (canceled)

42. A culture dish system with at least two culture dishes according to at least one of afore-mentioned claims, whereby fluids, media, or gas can be passed through at least two of no less than two culture dishes consecutively or at the same time, e.g. in a common circuit, and thus allow mediator exchange, and/or whereby cells in the compartments can migrate through at least two of no less than two culture dishes consecutively via pores in the walls, and/or pores in the membranes, and/or dedicated porous capillaries.

43. (canceled)

44. A method for using of a culture dish according to at least one of the afore-mentioned claims for cell and/or stem cell research, -culture, -expansion, -differentiation and -production, and/or for the production of substances by cells,

45. A method for using of a culture dish according to at least one of the afore-mentioned claims for cell-based therapy development and/or cell and/or stem cell preparation for regenerative medicine.

46. A method for using of a culture dish according to at least one of the afore-mentioned claims for immune cell maturation/sensitization and/or vaccine production.

47. A method for using of a culture dish according to at least one of the afore-mentioned claims as alternative method for animal research and/or pharmacologic-pharmaceutical studies.

48. (canceled)

49. A method for using of a culture dish according to at least one of the afore-mentioned claims for tumor- and cancer research, anti-tumor therapy development, and patient-individual anti cancer therapy control with patient tumor cells or patient cells.
